(19) United States
(12) Patent Application Publication
(10) Pub. No.: US 2005/0249822 A1
(43) Pub. Date: Nov. 10, 2005

(54) Administration of cisplatin by inhalation

(75) Inventors: Frank G. Pilkiewicz, Princeton Junction, NJ (US); Walter Perkins, Pennington, NJ (US); Beth Metzheiser, Cranbury, NJ (US)

Correspondence Address: FOLEY HOAG, LLP;PATENT GROUP, WORLD TRADE CENTER WEST, 155 SEAPORT BLVD, BOSTON, MA, 02110, US

(73) Assignee: Transave, Inc., Monmouth Junction, NJ (US)

(21) Appl. No.: 11/084,070

(22) Filed: Mar. 18, 2005

Related U.S. Application Data
(60) Provisional application No. 60/554,262, filed on Mar. 18, 2004.
(60) Provisional application No. 60/573,521, filed on May 21, 2004.

(51) Int. Cl. A61K 33/24

(52) U.S. Cl. 424/649

(57) ABSTRACT

Provided is a method for treating a patient having lung cancer. The method includes administering a lipid composition containing cisplatin to the patient's respiratory tract over the course of at least 2 treatment cycles. At least about 15 mg/m² of cisplatin is administered in each treatment cycle, and there is no more than 2 weeks between treatment cycles.

### RELATED APPLICATIONS

This application claims the benefit of priority to U.S. Provisional Application Ser. No. 60/554,262, filed Mar. 18, 2004, and U.S. Provisional Application Ser. No. 60/573,521, filed May 21, 2004; the entirety of which are hereby incorporated by reference.

### BACKGROUND OF THE INVENTION

The present invention relates to a method for treating cancer by delivering a therapeutically effective amount of a lipid composition containing a cytotoxic agent (e.g., cisplatin) to a patient's respiratory tract. The method allows clinicians to administer treatment cycles more frequently without the attendant side effects (e.g., nephrotoxicity, bone marrow toxicity) common to systemic administration of many cancer cytotoxic agents (e.g., cisplatin).

Bronchoalveolar Carcinoma (BAC) or alveolar cell carcinoma is a form of adenocarcinoma, a cell-type of non-small cell carcinoma of the lung which can be found throughout the respiratory tract. BAC represents approximately 10 to 25% of the adenocarcinoma of lung cases or 2-6% of all lung cancers and sometimes has a distinct presentation and biologic behavior. BAC is more common in women and in patients who do not smoke cigarettes than other histologic types of lung cancer.

BAC may present as a solitary peripheral nodule, a multifocal lesion, or a rapidly progressive form that appears as a diffuse infiltrate on chest radiograph. The cells secrete mucin and surfactant apoprotein which can lead to bronchorrhea, an excessive discharge of mucus from the air passages of the lungs. Bronchoalveolar cancer may present as a more diffuse lesion than other types of cancer. When it is discovered as a single mass on a patient's x-ray, this type of lung cancer has an excellent prognosis. Five year survival after surgery is in the 75-90 percent range. If, however, it is found in its diffuse form (meaning it has spread beyond a single mass), the prognosis is quite poor.

The management and prognosis are essentially the same as other types of non-small cell lung cancer. Surgery is the preferred treatment if the tumor can be resected. Radiation therapy and chemotherapy may be used in non-operable cases. Trials are underway to investigate treatments specific for bronchoalveolar carcinoma.

Carcinomatosis with lymphangitic spread, or Lymphangitis carcinomatosis (LC) refers to the diffuse infiltration and obstruction of pulmonary parenchymal lymphatic channels by tumor. Various neoplasms can cause lymphangitic carcinomatosis, but 80% are adenocarcinomas. The most frequent primary sites are the breast, lungs, colon, and stomach. Other sources include the pancreas, thyroid, cervix, prostate, larynx, and metastatic adenocarcinoma from an unknown primary.

LC occurs as a result of initial hematogenous spread of tumor to the lungs, with subsequent malignant invasion through the vessel wall into the pulmonary interstitium and lymphatics. The tumor then proliferates and spreads easily through these low resistance channels. Less commonly, direct infiltration occurs from contiguous mediastinal or hilar lymphadenopathy or from an adjacent primary bronchogenic carcinoma. Histopathologically, interstitial edema, interstitial fibrosis (secondary to a desmoplastic reaction as a result of tumor extension into adjacent pulmonary parenchyma), and tumor cells all can be seen. Metastatic adenocarcinoma accounts for 80% of cases. Most patients are middle-aged adults.

In the United States, LC represents 7% of all pulmonary metastases. Prevalence in postmortem studies is significantly higher than the incidence of radiologically detectable disease. Microscopic interstitial tumor invasion is seen in 56% of patients with pulmonary metastases. Prognosis for patients with LC is poor. Most patients survive only weeks or months.

Typically, chemotherapeutic treatment of lung cancers includes systemic administration of chemotherapeutic agents, e.g., cytotoxic agents, to the patients. Often such administration, e.g., intravenous administration, is associated with several adverse side effects including nephrotoxicity and bone marrow toxicity. For instance, systemic administration of cisplatin (cis-diamine-dichloroplatinum (II)) one of the more effective anti-tumor agents used in the systemic treatment of lung cancers, is often burdened by symptoms such as nephrotoxicity in the patient. The nephrotoxicity limits the frequency in which clinicians can administer cisplatin to the patient. In fact, successive treatment cycles of cisplatin typically require three weeks or more between treatment cycles to prevent blood levels of cisplatin from reaching those correlated with nephrotoxicity. Since chemotherapeutic regimens typically require five or more treatment cycles, the delay between treatment cycles lengthens the time needed for the overall chemotherapeutic regimen. The prolonged time periods for systemic administration of cisplatin lead to increased patient discomfort and inconvenience, and may lead to decreased patient compliance.

Accordingly, new methods for treating patients suffering from lung cancer by inhalation administration of cisplatin that allow significant local concentrations of drug to be attained by shortening of the time periods needed between treatment cycles are desirable. Such methods preferably also overcome the rapid clearance of cisplatin from the lung that typically plague inhalation administration of therapeutic agents.

### SUMMARY OF THE INVENTION

In one aspect, the present invention features a method for treating a patient having cancer, comprising administering a lipid composition comprising cisplatin to the patient's respiratory tract over the course of at least 2 treatment cycles, wherein: at least about 15 mg/m² of cisplatin is administered in each treatment cycle; and there is no more than 2 weeks between treatment cycles.

In a preferred embodiment, there is no more than 1 week between treatment cycles. In another preferred embodiment, the method comprises at least 3 treatment cycles. In another preferred embodiment, the method includes at least 4 treatment cycles, and more preferably, at least 5 treatment cycles.

In another preferred embodiment, the cisplatin to lipid ratio of the lipid composition is from about 1:50 to about 1:5 by weight. In a further embodiment, the cisplatin to lipid ratio of the lipid composition is from about 1:50 to about 1:10 by weight. In a further embodiment, the cisplatin to lipid ratio of the lipid composition is from about 1:25 to about 1:15 by weight.

In another preferred embodiment, the lipid composition comprises a lipid selected from the group consisting of egg phosphatidylcholine (EPC), egg phosphatidylglycerol (EPG), egg phosphatidylinositol (EPI), egg phosphatidylserine (EPS), egg phosphatidylethanolamine (EPE), egg phosphatidic acid (EPA), soy phosphatidylcholine (SPC), soy phosphatidylglycerol (SPG), soy phosphatidylserine (SPS), soy phosphatidylinositol (SPI), soy phospatidylethanolamine (SPE), soy phosphatic acid (SPA), hydrogenated egg phosphatidylcholine (HEPC), hydrogenated egg phosphatidylglycerol (HEPG), hydrogenated egg phosphatidylinositol (HEPI), hydrogenated egg phosphatidylserine (HEPS), hydrogenated egg phosphatidylethanolamine (HEPE), hydrogenated egg phosphatidic acid (HEPA), hydrogenated soya phosphatidylcholine (HSPC), hydrogenated soy phosphafidylglycerol (HSPG), hydrogenated soy phosphatidylserine (HSPS), hydrogenated soy phosphatidylinositol (HSPI), hydrogenated soy phospatidylethanolamine (HSPE), hydrogenated soy phosphatic acid (HSPA), dipalmitoylphosphatidylcholine (DPPC), dimyristoylphosphatidycholine (DMPC), dimyristoylphosphatidylglycerol (DMPG), dipalmitoylphosphatidylglycerol (DPPG), distearoylphosphatidylcholine (DSPC), distearoylphosphatidylglycerol (DSPG), dioleylphosphatidyl-ethanolarnine (DOPE), palmitoylstearoylphosphatidyl-choline (PSPC), palmitoylstearolphosphatidylglycerol (PSPG), mono-oleoyl-phosphatidylethanolamine (MOPE), cholesterol, cholesterol hemi-succinate, cholesterol hydrogen sulfate, cholesterol sulfate, ergosterol, ergosterol hemi-succinate, ergosterol hydrogen sulfate, ergosterol sulfate, lanosterol, lanosterol hemi-succinate, lanosterol hydrogen sulfate, lanosterol sulfate, and mixtures thereof. In a further embodiment, the lipid composition comprises DPPC. In a further embodiment, the lipid composition comprises cholesterol. In still a further embodiment, the lipid component of the lipid composition comprises from about 50 to about 65 mol % of DPPC and about 35 to about 50 mol % cholesterol.

In another preferred embodiment, the lipid composition further comprises an aqueous component. In a further embodiment, there is at least 80% by weight of the aqueous component in the lipid composition.

In another preferred embodiment, the lipid composition is administered as an aerosol. In another preferred embodiment, the lipid composition is administered with a nebulizer at a flow rate of at least about 0.15 mL/min.

In another preferred embodiment, the lipid composition comprises one or more liposomes.

In another preferred embodiment, the cancer is a lung cancer. In a further embodiment, the lung cancer is selected from the group consisting of bronchoalveolar carcinoma and carcinomatosis with lymphangitic spread. In a further embodiment, the lung cancer is bronchoalveolar carcinoma.

In another aspect, the present invention features a method for treating a patient having bronchoalveolar carcinoma, comprising administering a lipid composition comprising cisplatin to the patient's respiratory tract over the course of at least 5 treatment cycles, wherein at least about 15 mg/m² of cisplatin is administered in each treatment cycle; there is no more than 2 weeks between treatment cycles; the lipid composition comprises from about 50 to about 65 mol % of DPPC and about 35 to about 50 mol % cholesterol; and the cisplatin to lipid ratio is from about 1:25 to about 1:15 by weight.

In a preferred embodiment, the lipid composition further comprises at least 80% by weight of aqueous component and the lipid composition is administered with a nebulizer.

In a preferred embodiment, the lipid composition is administered with a nebulizer at a flow rate of at least about 0.15 mL/min.

These embodiments of the present invention, other embodiments, and their features and characteristics, will be apparent from the description and claims that follow.

### DETAILED ESCRIPTION OF THE INVENTION

Definitions

For convenience, before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

An “active platinum” compound is a compound containing coordinated platinum and having antineoplastic activity. Active platinum compounds include, for example, cisplatin, carboplatin, and DACH-platinum compounds such as oxaplatin.

A “patient,” “subject” or “host” to be treated by the subject method may mean either a human or non-human animal.

The term “therapeutic effect” is art-recognized and refers to a local or systemic effect in animals, particularly mammals, and more particularly humans caused by a pharmacologically active substance. The phrase “therapeutically-effective amount” means that amount of a substance that produces some desired local or systemic effect at a reasonable benefit/risk ratio applicable to any treatment. The therapeutically effective amount of a substance will vary depending upon the subject and disease condition being treated, the weight and age of the subject, the severity of the disease condition, the manner of administration and the like, which can readily be determined by one of ordinary skill in the art.

The term “treating” is art-recognized and refers to curing as well as ameliorating at least one symptom of a condition or disease or preventing the occurrence of a condition or disease.

“Treatment cycle” means the time period in which a given dose of cisplatin is to be administered to a patient. Treatment cycles may encompass one or more sessions where the patient is actively being administered the liposomal composition containing cisplatin. Such sessions may be administered over the course of four days or less, but more preferably is administered over one or two days.

General

Provided is a method for treating lung cancer by delivering a therapeutically effective amount of a lipid composition containing cisplatin to a patient's respiratory tract. The method allows more intensive chemotherapeutic treatment of patients. In particular, with the methods of the invention clinicians may safely administer treatment cycles with cisplatin to patients more frequently. Consequently, less time is needed to complete the entire therapeutic regimen.

The method includes administering a lipid composition containing cisplatin to the patient's respiratory tract over the course of at least two treatment cycles. During each treatment cycle, at least about 15 mg/m² of cisplatin is administered to the patient's respiratory tract, and the time period between treatment cycles is no more than two weeks. As used herein the time period between the treatment cycles refers to the time period between initiation of each consecutive treatment cycle. The lipid composition administered is a liposomal/lipid complex composition containing cisplatin which may be, for example, combined with an aqueous component, e.g., saline, and administered as an aerosol.

In general, the doses of cisplatin will be chosen by a physician based on the age, physical condition, weight and other factors known in the medical arts. In each treatment cycle preferred dosages are between approximately 15 mg/m² and approximately 60 mg/m². Generally there is no more than two weeks between treatment cycles, and preferably, there is no more than one week between treatment cycles.

The method provides for a more intensive chemotherapeutic regimen for lung cancer treatment due to the localized delivery of cisplatin to the respiratory tract. Clinicians may administer more drug to lung cancer patients with greater frequency of treatment cycles because the method minimizes systemic exposure of non-cancerous cells in the body to the toxic effects of cisplatin. The patient's,propensity for nephrotoxicity, which typically limits the frequency of treatment cycles for systemically administered cisplatin, is diminished.

The method also overcomes the drawbacks generally associated with inhalation administration of drugs due to the lipid composition. The lipid composition (particularly liposome-based compositions) serves to protect the cisplatin as it is delivered to its target site, and protects non-cancerous tissue from being exposed to the cytotoxic effects of the drug. In addition, the lipid composition facilitates adherence of the composition to the lungs, and slows the release of the drug, and thereby diminishes the rapid clearance typically associated with inhalational administration. Moreover, the compositions are sufficiently stable in the lungs to allow the formulation to remain effective for a therapeutically useful time period.

The method includes a lipid composition that has a very high cisplatin to lipid ratio. The bioactive agent to lipid ratio seen in the present invention is between about 1:5 by weight and about 1:50 by weight. More preferably, the bioactive agent to lipid ratio seen is between about 1:10 by weight and about 1:30 by weight. Most preferably, the bioactive agent to lipid ratio seen is between about 1:15 by weight and about 1:25 by weight. When formulated with an aqueous component for administration with a nebulizer, the cisplatin may be present in the final formulation at from about 0.5 mg/mL to about 1.7 mg/mL, and is preferably present at from about 0.8 to about 1.3 mg/mL.

In addition to the lipid component, cisplatin, and optional aqueous component, the lipid composition may also contain commonly used pharmaceutically acceptable excipients (including solvents, salts and buffers), preservatives and surfactants.

The lipid compositions can include liposomes, lipid complexes, lipid clathrates and proliposomes, i.e., compositions which can form liposomes in vitro or in vivo when contacted with water. Compositions are preferably adopted for use by inhalation, and more preferably for use in an inhalation delivery device for the composition's administration. The inhalation system can be used for the treatment of lung cancers in both man and animal.

Methods of Preparing the Lipid Compositions

The lipid composition is preferably formed as described in co-pending U.S. patent application Ser. No. 10/634,144, filed Aug. 4, 2003, which is hereby incorporated by reference in its entirety. Briefly, the lipid complex can be formed by mixing cisplatin with an appropriate lipid dissolved or suspended in a solvent (e.g., ethanol) and subjecting the mixture to one or more cycles have two separate temperatures. The process is believed to be in the form of an active platinum compound aggregate.

In aqueous solution, cisplatin forms large crystalline aggregates with a crystal diameter of greater than a few microns. In the presence of an amphipathic matrix system, such as a lipid bilayer, cisplatin complexes with the lipid. For example, the complexes may be formed in the hydrocarbon core region of a lipid bilayer. During the warming cycle of the process, it is believed that cisplatin is returned to solution at a greater rate in aqueous regions of the process mixture than in the bilayers. As a result of applying more than one cool/warm cycle, cisplatin accumulates further in the lipid bilayers. Without limiting the invention to the proposed theory, experimentation indicates that the cisplatin complexes cause the immediate surroundings of the interfacial bilayer region to be more hydrophobic and compact. This results in a high level of entrapment of active platinum compound as cooling and warming cycles are repeated.

The formulation has a markedly high entrapment percentage of cisplatin. The entrapment has been shown, in some cases, to reach upto about 20, 30, 40, 50, 60, 70, 80, or about 90%. This amount is far higher than the most efficient entrapment expected from a conventional aqueous entrapment which is approximately 2-10% entrapment.

The process includes combining cisplatin with a hydrophobic matrix carrying system (lipid/solvent mixture) and cycling the solution between a warmer and a cooler temperature. Preferably, the cycling is performed more than one time. More preferably, the step is performed two or more times, or three or more times. The cooler temperature portion of cycle can, for example, use a temperature from about −25° C. and about 25° C. More preferably, the step uses a temperature from about −5 and about 5° C. or between about 1 and about 5° C. For manufacturing convenience, and to be sure the desired temperature is established, the cooler and warmer steps can be maintained for a period of time, such as approximately from about 5 to about 300 minutes or about 30 to about 60 minutes. The step of warming includes warming the reaction vessel to from about 4 and about 70° C. More preferably, the step of warming comprises heating the reaction vessel to from about 45 to about 55° C. The above temperature ranges are particularly preferred for use with lipid compositions containing predominantly dipalmitoylphosphatidycholine (DPPC) and cholesterol.

Another way to consider the temperature cycling is in terms of the temperature differential between the warmer and the cooler steps of the cycle. This temperature differential can be, for example, about 25° C. or more, such as a differential from about 25 to about 70° C., preferably a differential from about 40 to about 55° C. The temperatures of the cooler and higher temperature steps are selected on the basis of increasing entrapment of active platinum compound. Without being limited to theory, it is believed that it is useful to select an upper temperature effective to substantially increase the solubility of active platinum compound in the processed mixture. Preferably, the warming step temperature is about 50° C. or higher. The temperatures can also be selected to be below and above the transition temperature for a lipid in the lipid composition.

The temperatures appropriate for the method describe above may, in some cases, vary with the lipid composition used in the method, as can be determined by ordinary experimentation.

Experimental results strongly indicate that encapsulation was achieved predominantly by capturing cisplatin during formation of liposomal vesicles. The results further indicate the physical state of cisplatin to be solid (aggregates) or lipid bound since the concentration of cisplatin is much higher than the solubility limit. Results further indicate that process does not require freezing the compositions, but that cooling to temperature higher than freezing can produce superior results. Results further indicated that an entrapment efficiency achieved by 3 cycles was similar to that achieved by 6 cycles of cooling and warming cycles, which indicated that 3 cycles of temperature treatment was sufficient to achieve highly preferred levels of entrapment.

Results further indicate that the process can be scaled-up while increasing process efficiency in entrapping cisplatin. Thus, the invention further provides processes that are conducted to provide an amount adapted for total administration (in appropriate smaller volume increments) of about 200 or more mLs, about 400 or more mLs, or about 800 or more mLs. All else being the same, it is believed that the larger production volumes generally achieve increased efficiency over smaller scale processes. While such volume is that appropriate for administration, it will be recognized that the volume can be reduced for storage.

Results further indicate that the lipid-complexed cisplatin made by this method can retain entrapped cisplatin with minimal leakage for over one year. This is a further demonstration of the uniqueness in the formulation, indicating that the cisplatin is bound within the liposome structure and not free to readily leak out.

Lipids

The lipids used in the compositions of the present invention can be synthetic, semi-synthetic or naturally-occurring lipids, and typically include phospholipids and sterols. In terms of phosholipids, they could include such lipids as egg phosphatidylcholine (EPC), egg phosphatidylglycerol (EPG), egg phosphatidylinositol (EPI), egg phosphatidylserine (EPS), phosphatidylethanolamine (EPE), and phosphatidic acid (EPA); the soya counterparts, soy phosphatidylcholine (SPC); SPG, SPS, SPI, SPE, and SPA; the hydrogenated egg and soya counterparts (e.g., HEPC, HSPC), other phospholipids made up of ester linkages of fatty acids in the 2 and 3 of glycerol positions containing chains of 12 to 26 carbon atoms and different head groups in the 1 position of glycerol that include choline, glycerol, inositol, serine, ethanolamine, as well as the corresponding phosphatidic acids. The chains on these fatty acids can be saturated or unsaturated, and the phospholipid may be made up of fatty acids of different chain lengths and different degrees of unsaturation. In particular, the compositions of the formulations can include DPPC, a major constituent of naturally-occurring lung surfactant. Other examples include dimyristoylphosphatidycholine (DMPC) and dimyristoylphosphatidylglycerol (DMPG), dipalmitoylphosphatidylglycerol (DPPG) distearoylphosphatidylcholine (DSPC) and distearoylphosphatidylglycerol (DSPG), dioleylphosphatidyl-ethanolamine (DOPE) and mixed phospholipids like palmitoylstearoylphosphatidyl-choline (PSPC) and palmitoylstearolphosphatidylglycerol (PSPG), and single acylated phospholipids like mono-oleoyl-phosphatidylethanolamine (MOPE).

The sterols can include, cholesterol, esters of cholesterol including cholesterol hemi-succinate, salts of cholesterol including cholesterol hydrogen sulfate and cholesterol sulfate, ergosterol, esters of ergosterol including ergosterol hemi-succinate, salts of ergosterol including ergosterol hydrogen sulfate and ergosterol sulfate, lanosterol, esters of lanosterol including lanosterol hemi-succinate, salts of lanosterol including lanosterol hydrogen sulfate and lanosterol sulfate.

In a preferred embodiment of the invention the lipid composition contains 50 to 100 mol % DPPC and 0 to 50 mol % cholesterol. More preferably, the lipid complex contains 50 to 65 mol % DPPC and 35 to 50 mol % cholesterol.

Inhalation Devices

The inhalation delivery device of the inhalation system can be a nebulizer, a metered dose inhaler (MDI) or a dry powder inhaler (DPI). The device can contain and be used to deliver a single dose of the lipid compositions or the device can contain and be used to deliver multi-doses of the lipid compositions of the present invention.

A nebulizer type inhalation delivery device can contain the compositions of the present invention as a solution, usually aqueous, or a suspension. In generating the nebulized spray of the compositions for inhalation, the nebulizer type delivery device may be driven ultrasonically, by compressed air, by other gases, electronically or mechanically (including, for example, a vibrating porous membrane). The ultrasonic nebulizer device usually works by imposing a rapidly oscillating waveform onto the liquid film of the formulation via an electrochemical vibrating surface. At a given amplitude the waveform becomes unstable, whereby it disintegrates the liquids film, and it produces small droplets of the formulation. The nebulizer device driven by air or other gases operates on the basis that a high pressure gas stream produces a local pressure drop that draws the liquid formulation into the stream of gases via capillary action. This fine liquid stream is then disintegrated by shear forces. The nebulizer may be portable and hand held in design, and may be equipped with a self contained electrical unit. The nebulizer device can consist of a nozzle that has two coincident outlet channels of defined aperture size through which the liquid formulation can be accelerated. This results in impaction of the two streams and atomization of the formulation. The nebulizer may use a mechanical actuator to force the liquid formulation through a multiorifice nozzle of defined aperture size(s) to produce an aerosol of the formulation for inhalation. In the design of single dose nebulizers, blister packs containing single doses of the formulation may be employed.

In the present invention the nebulizer is employed to ensure the sizing of aqueous droplets containing the drug-lipid particles is optimal for positioning of the particle within, for example, the lungs. Typical droplet sizes for the nebulized lipid composition are from about 1 to about 5 microns.

For use with the nebulizer, the lipid composition preferably contains an aqueous component. Typically there is at least about 80% by weight and preferably, at least about 90% by weight of the aqueous component in the lipid composition to be administered with a nebulizer. The aqueous component may include for example, saline. In addition, the aqueous component may include up to about 20% by weight of an aqueous compatible solvent such as ethanol.

Total administration time using a nebulizer will depend on the flow rate and the concentration of the cisplatin in the lipid composition. Variation of the total administration time is within the purview of those of ordinary skill in the art. Generally, the flow rate of the nebulizer will be at least about 0.15 mL/min, for example, a flow rate of about 0.2 mL/min is typical. By way of example, administration of a dose of about 24 mg/m² of cisplatin using a lipid composition having a concentration of about 1 mg/mL of cisplatin would be about 4 hours (assuming a patient's body surface area is about 2 m²). This administration time may, for example, be split into two administration sessions given over the course of one or two days to complete one treatment cycle.

In alternative embodiments, a metered dose inhalator (MDI) can be employed as the inhalation delivery device of the inhalation system. This device is pressurized (pMDI) and its basic structure consists of a metering valve, an actuator and a container. A propellant is used to discharge the formulation from the device. The composition can consist of particles of a defined size suspended in the pressurized propellant(s) liquid, or the composition can be in a solution or suspension of pressurized liquid propellant(s). The propellants used are primarily atmospheric friendly hydroflourocarbons (HFCs) such as 134a and 227. Traditional chloroflourocarbons like CFC-11, 12 and 114 are used only when essential. The device of the inhalation system may deliver a single dose via, e.g., a blister pack, or it may be multi dose in design. The pressurized metered dose inhalator of the inhalation system can be breath actuated to deliver an accurate dose of the lipid based formulation. To insure accuracy of dosing, the delivery of the formulation may be programmed via a microprocessor to occur at a certain point in the inhalation cycle. The MDI may be portable and hand held.

In another alternative embodiment, a dry powder inhalator (DPI) can be used as the inhalation delivery device of the inhalation system. This device's basic design consists of a metering system, a powdered composition and a method to disperse the composition. Forces like rotation and vibration can be used to disperse the composition. The metering and dispersion systems may be mechanically or electrically driven and may be microprocessor programmable. The device may be portable and hand held. The inhalator may be multi or single dose in design and use such options as hard gelatin capsules, and blister packages for accurate unit doses. The composition can be dispersed from the device by passive inhalation; i.e., the patient's own inspiratory effort, or an active dispersion system may be employed. The dry powder of the composition can be sized via processes such as jet milling, spray dying and supercritical fluid manufacture. Acceptable excipients such as the sugars mannitol and maltose may be used in the preparation of the powdered formulations. These are particularly important in the preparation of freeze dried liposomes and lipid complexes. These sugars help in maintaining the liposome's physical characteristics during freeze drying and minimizing their aggregation when they are administered by inhalation. The hydroxyl groups of the sugar may help the vesicles maintain their tertiary hydrated state and help minimize particle aggregation.

The inventive method is particularly well-suited for the treatment of lung cancers, particularly, bronchoalveolar carcinoma, or carcinomatosis with lymphangitic spread. In addition, both primary and metastatic lung cancers are excellent candidates for the method of the invention.

Dosages

The dosage of any composition of the present invention will vary depending on the symptoms, age and body weight of the patient, the nature and severity of the disorder to be treated or prevented, the route of administration, and the form of the supplement. Any of the subject formulations may be administered in a single dose or in divided doses. Dosages for the compounds of the present invention may be readily determined by techniques known to those of skill in the art or as taught herein. Also, the present invention contemplates mixtures of more than one subject compound, as well as other therapeutic agents. Further, the present invention contemplates administration of the therapeutic agent that is contained in a subject coordination complex (or a related agent) in conjunction with the complex itself to increase the ratio of the therapeutic agent to the coordination complex formed upon release of the therapeutic agent,

In certain embodiments, the dosage of the subject compounds will generally be in the range of about 0.01 ng to about 10 g per kg body weight, specifically in the range of about 1 ng to about 0.1 g per kg, and more specifically in the range of about 100 ng to about 10 mg per kg.

An effective dose or amount, and any possible affects on the timing of administration of the formulation, may need to be identified for any particular compound of the present invention. This may be accomplished by routine experiment as described herein, using one or more groups of animals (preferably at least 5 animals per group), or in human trials if appropriate. The effectiveness of any compound and method of treatment or prevention may be assessed by administering the supplement and assessing the effect of the administration by measuring one or more indices associated with the neoplasm of interest, and comparing the post-treatment values of these indices to the values of the same indices prior to treatment.

The precise time of administration and amount of any particular compound that will yield the most effective treatment in a given patient will depend upon the activity, pharmacokinetics, and bioavailability of a particular compound, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

While the subject is being treated, the health of the patient may be monitored by measuring one or more of the relevant indices at predetermined times during a 24-hour period. Treatment, including supplement, amounts, times of administration and formulation, may be optimized according to the results of such monitoring. The patient may be periodically reevaluated to determine the extent of improvement by measuring the same parameters, the first such reevaluation typically occurring at the end of four weeks from the onset of therapy, and subsequent reevaluations occurring every four to eight weeks during therapy and then every three months thereafter. Therapy may continue for several months or even years, with a minimum of one month being a typical length of therapy for humans. Adjustments to the amount(s) of agent administered and possibly to the time of administration may be made based on these reevaluations.

Treatment may be initiated with smaller dosages which are less than the optimum dose of the compound. Thereafter, the dosage may be increased by small increments until the optimum therapeutic effect is attained.

The combined use of several compounds of the present invention, or alternatively other chemotherapeutic agents, may reduce the required dosage for any individual component because the onset and duration of effect of the different components may be complimentary. In such combined therapy, the different active agents may be delivered together or separately, and simultaneously or at different times within the day.

Toxicity and therapeutic efficacy of subject compounds may be determined by standard pharmaceutical procedures in cell cultures or experimental animals, e.g., for determining the LD₅₀ and the ED₅₀. Compositions that exhibit large therapeutic indices are preferred. Although compounds that exhibit toxic side effects may be used, care should be taken to design a delivery system that targets the compounds to the desired site in order to reduce side effects.

The data obtained from the cell culture assays and animal studies may be used in formulating a range of dosage for use in humans. The dosage of any supplement, or alternatively of any components therein, lies preferably within a range of circulating concentrations that include the ED₅₀ with little or no toxicity. The dosage may vary within this range depending upon the dosage form employed and the route of administration utilized. For agents of the present invention, the therapeutically effective dose may be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information may be used to more accurately determine useful doses in humans. Levels in plasma may be measured, for example, by high performance liquid chromatography.

Kits

This invention also provides kits for conveniently and effectively implementing the methods of this invention. Such kits comprise any of the compounds of the present invention or a combination thereof, and a means for facilitating compliance with methods of this invention. Such kits provide a convenient and effective means for assuring that the subject to be treated takes the appropriate active in the correct dosage in the correct manner. The compliance means of such kits includes any means which facilitates administering the actives according to a method of this invention. Such compliance means include instructions, packaging, and dispensing means, and combinations thereof. Kit components may be packaged for either manual or partially or wholly automated practice of the foregoing methods. In other embodiments involving kits, this invention contemplates a kit including compositions of the present invention, and optionally instructions for their use.

The following examples further illustrate the present invention, but of course, should not be construed as in any way limiting its scope.

### EXEMPLIFICATION

### Example 1

70 mg of DPPC and 28 mg of cholesterol were dissolved in 1 mL of ethanol and added to 10 mL of 4 mg/mL cisplatin in 0.9% saline solution. An aliquot (50%) of the sample was treated by 3 cycles of cooling to 4° C. and warming to 50° C. The aliquot, in a test tube, was cooled by refrigeration, and heated in a water bath. The resulting unentrapped cisplatin (free cisplatin) was washed by dialysis. The remainder of the sample was not treated by temperature cycles and directly washed by dialysis. Table 1 presents the percentage entrapment of cisplatin with and without cooling an warming cycles.

TABLE 1
Cisplatin percentage entrapment.

| | Final Concentration of | |
|---|---|---|
| | cisplatin, μg/ml | % Entrapment |
| Lipid-complexed cisplatin | 56 | 1.4 |
| without cooling and | | |
| warming cycles | | |
| Lipid-complexed cisplatin | 360 | 9.0 |
| after cooling and warming | | |
| cycles | | |

### Example 2

1.0 g of DPPC and 0.4 g of cholesterol were dissolved in 6 mL of ethanol. 60 mg of cisplatin was dissolved in 10 mL of 0.9% saline solution at 65° C. 1 mL of the resultant lipid mixture solution was added to 10 mL of the resultant cisplatin solution. The lipid/cisplatin suspension was cooled to approximately 4° C. and held at that temperature for 20 minutes and warmed to 50° C. and held at that temperature for 20 minutes. Ethanol was removed by bubbling N₂ gas into the suspension during the warming period. The cooling and warming steps were repeated 5 further times. The concentration of total cisplatin was 5.8 mg/mL with 91.6% entrapped cisplatin and drug:lipid ratio (by weight) of 1:26.

### Example 3

A liposomal formulation was prepared using phosphatidylcholine (PC) and cholesterol (in a 57:43 mol ratio). 0.55 mmoles of PC and 0.41 mmoles of cholesterol were dissolved in 2 mL ethanol and added to 20 mL of 4 mg/mL cisplatin solution. An aliquot (50%) of each sample was treated by 3 cycles of cooling and warming and then washed by dialysis. Another part of each sample was directly washed by dialysis. Entrapment was estimated from the ratio of final concentration and initial concentration.

TABLE 2
Entrapment and drug to lipid ratios for
cisplatin with various phophatidylcholines.

| | No Cooling and Warming | | | Cooling and Warming | | |
|---|---|---|---|---|---|---|
| | Final | | | Final | | |
| | [Cisplatin] | % | Drug:Lipid | [Cisplatin] | % | Drug:Lipid |
| PC | (mg/mL) | Entrapment | (by weight) | (mg/mL) | Entrapment | (by weight) |
| DOPC | 0.16 | 4.0 | 1:142 | 0.21 | 5.3 | 1:108 |
| EggPC | 0.09 | 2.3 | 1:247 | 0.12 | 3.0 | 1:185 |
| DMPC | 0.15 | 3.8 | 1:123 | 0.24 | 6.0 | 1:77 |
| DPPC | 0.17 | 4.3 | 1:115 | 0.85 | 21.3 | 1:23 |
| HSPC | 0.11 | 2.8 | 1:202 | 0.23 | 5.8 | 1:97 |
| DSPC | 0.10 | 2.5 | 1:184 | 0.58 | 14.5 | 1:32 |

### Example 4

A lipid formulation (DPPC:cholesterol in a ratio of 5:2 w/w) was dissolved in ethanol and added to a cisplatin solution. Part of the formulation was treated by cycles of cooling to 4° C. and warming to 55° C. cycles while part was not treated thus. The lipid/cisplatin suspension was then washed by dialysis.

TABLE 3
Concentration of cisplatin with and without cooling
and warming cycles.

| Starting | | | |
|---|---|---|---|
| concentration of | Concentration of | Cooling & | Total concentration |
| Cisplatin solution | lipids | warming | of Cisplatin |
| (mg/mL) | (mg/mL) | cycles | (mg/mL) |
| 0.2 | 1.4 | No | Not Detectable |
| 0.2 | 1.4 | Yes | Not Detectable |
| 4.0 | 28 | No | 0.22 |
| 4.0 | 28 | Yes | 0.46 |

### Example 5

Dosing Schedule

Patients are dosed with a jet nebulizer (Pari LC Star) which is filled with up to about 7 mL of the lipid composition (containing about 1 mg/mL of cisplatin) which is formulated with saline. The flow rate of the lipid composition from the nebulizer is about 0.2 mL/min. At this rate, for example, administration of about 4 mL of the lipid composition takes about 20 minutes. Table 4 indicates the dosing schedule.

TABLE 4
Dosing schedule.

| | Dose/Treatment | Frequency of | |
|---|---|---|---|
| | Cycle | Treatment Cycles | # of Treatment |
| Patient | (mg/m²) | (week(s)) | Cycles |
| 1 | 1.5 | 3 | 6 (i.e., 18 weeks) |
| 2 | 3.0 | 3 | 6 |
| 3 | 6.0 | 3 | 6 |
| 4 | 12.0 | 3 | 6 |
| 5 | 24.0 | 3 | 6 |
| 6 | 48.0 | 3 | 6 |
| 7 | 24.0 | 2 | 6 (i.e., 12 weeks) |
| 8 | 36.0 | 2 | 6 |
| 9 | 48.0 | 2 | 6 |
| 10 | 24.0 | 1 | 12 (i.e., 3 months) |

Patient numbers 1, 3, and 9, of the ongoing study have shown stabilization (i.e., no further tumor growth or tumor growth of less than 20%).

### INCORPORATION BY REFERENCE

All of the patents and publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method for treating a patient having cancer, comprising:
administering a lipid composition comprising cisplatin to the patient's respiratory tract over the course of at least 2 treatment cycles, wherein:
at least about 15 mg/m² of cisplatin is administered in each treatment cycle; and
there is no more than 2 weeks between treatment cycles.

2. The method of claim 1, wherein there is no more than 1 week between treatment cycles.

3. The method of claim 1, wherein the method comprises at least 3 treatment cycles.

4. The method of claim 1, wherein the method comprises at least 4 treatment cycles.

5. The method of claim 1, wherein the method comprises at least 5 treatment cycles.

6. The method of claim 1, wherein the cisplatin to lipid ratio of the lipid composition is from about 1:50 to about 1:5 by weight.

7. The method of claim 6, wherein the cisplatin to lipid ratio of the lipid composition is from about 1:50 to about 1:10 by weight.

8. The method of claim 7, wherein the cisplatin to lipid ratio of the lipid composition is from about 1:25 to about 1:15 by weight.

9. The method of claim 1, wherein the lipid composition comprises DPPC.

10. The method of claim 1, wherein the lipid composition comprises cholesterol.

11. The method of claim 1, wherein the lipid component of the lipid composition comprises from about 50 to about 65 mol % of DPPC and about 35 to about 50 mol % cholesterol.

12. The method of claim 1, wherein the lipid composition further comprises an aqueous component.

13. The method of claim 12, wherein there is at least 80% by weight of the aqueous component in the lipid composition.

14. The method of claim 1, wherein the lipid composition is administered as an aerosol.

15. The method of claim 14, wherein the lipid composition is administered with a nebulizer at a flow rate of at least about 0.15 mL/min.

16. The method of claim 1, wherein the lipid composition comprises one or more liposomes.

17. The method of claim 1, wherein the cancer is a lung cancer.

18. The method of claim 17, wherein the lung cancer is selected from the group consisting of bronchoalveolar carcinoma and carcinomatosis with lymphangitic spread.

19. The method of claim 18, wherein the lung cancer is bronchoalveolar carcinoma.

20. A method for treating a patient having bronchoalveolar carcinoma, comprising:
administering a lipid composition comprising cisplatin to the patient's respiratory tract over the course of at least 5 treatment cycles, wherein:
at least about 15 mg/m² of cisplatin is administered in each treatment cycle;
there is no more than 2 weeks between treatment cycles;
the lipid composition comprises from about 50 to about 65 mol % of DPPC and about 35 to about 50 mol % cholesterol; and
the cisplatin to lipid ratio is from about 1:25 to about 1:15 by weight.

21. The method of claim 20, wherein the lipid composition further comprises at least 80% by weight of aqueous component and the lipid composition is administered with a nebulizer.

22. The method of claim 21, wherein lipid composition is administered with a nebulizer at a flow rate of at least about 0.15 mL/min.
